(19) United States
(12) Patent Application Publication
(10) Pub. No.: US 2022/0381718 A1
(43) Pub. Date: Dec. 1, 2022

(54) BIPHASIC COATINGS WITH CHEMICAL SENSING, AND METHODS OF MAKING AND USING THE SAME

(71) Applicant: HRL Laboratories, LLC, Malibu, CA (US)

(72) Inventors: Adam GROSS, Santa Monica, CA (US); John VAJO, West Hills, CA (US); Jason GRAETZ, Calabasas, CA (US)

(21) Appl. No.: 17/680,195

(22) Filed: Feb. 24, 2022

Related U.S. Application Data
(60) Provisional application No. 63/194,312, filed on May 28, 2021.

(51) Int. Cl. G01N 27/04

(52) U.S. Cl. CPC G01N 27/041; G01N 2033/0096

(57) ABSTRACT

Some variations provide a system for sensing a chemical active in a coating, the system comprising: a coating disposed on a substrate; a chemical active contained within the coating, wherein the chemical active is mobile within the coating, and wherein the chemical active is ionically and/or electrically conductive; a first electrode and a second electrode configured to measure AC impedance within the coating; and an electrical meter configured in electrical communication with the first and second electrodes to read a signal corresponding to the AC impedance. Some methods comprise: pressing electrodes against the coating; reading out an impedance value; and converting the impedance value to a concentration of the chemical active in the coating. Other methods comprise: adding a solvent to a coating surface; pressing electrodes against a surface region; reading out an impedance value; and converting the impedance value to a concentration of the chemical active in the coating.

### PRIORITY DATA

This patent application claims priority to U.S. Provisional Patent App. No. 63/194,312, filed on May 28, 2021, which is hereby incorporated by reference herein.

### FIELD OF THE INVENTION

The present invention generally relates to coatings configured with chemical sensing of active components within the coating, and methods of making and using the same.

### BACKGROUND OF THE INVENTION

Coatings are prevalent in the world today. Some coatings are designed to physically or chemically protect an underlying substrate. Certain coatings are designed to contain chemical actives, with various functions. A “chemical active” is a chemical that has one or more desirable activities for a chemical, electrochemical, electrical, or biological reaction. An exemplary chemical active is an antimicrobial agent.

Coatings containing chemical actives are only efficacious when the amount of chemical active is above a critical level which depends on the specific function. When coatings are in the environment, as is often the case, chemical actives tend to leech out. Conventionally, there is no facile way to measure whether a chemical active is above a critical level for efficacy within a coating.

Current methods to ensure there is a sufficient amount of a chemical active are inferior. In one approach, a surface is periodically recoated without knowledge that the chemical actives have been depleted. Another approach utilizes a chemical test kit on the coating. This approach can damage the coating and is also time-consuming and inconvenient. Another approach involves swabbing the coating and taking a sample to an off-site laboratory. This method has an especially slow response time of days to weeks or even longer.

What is needed is a real-time indication of when a chemical active, such as an antimicrobial agent, is no longer functioning as desired. It would be particularly beneficial to be able to sense when biological and chemical self-cleaning or self-decontaminating coatings are still effective, for example.

### SUMMARY OF THE INVENTION

Some variations of the invention provide a system for sensing a chemical active in a coating, the system comprising:

a coating disposed on a substrate;

a chemical active contained within the coating, wherein the chemical active is mobile within the coating, and wherein the chemical active is ionically and/or electrically conductive;

a first electrode and a second electrode configured to measure AC impedance within the coating; and

an electrical meter configured in electrical communication with the first and second electrodes to read a signal corresponding to the AC impedance.

In some embodiments, the coating is a polymer. The polymer may contain at least a first phase that is continuous and a second phase that is discrete or continuous. The first phase and the second phase may be phase-separated on an average length scale of phase separation selected from about 10 nanometers to about 1 millimeter, such as selected from about 100 nanometers to about 25 microns. Typically, the first phase and the second phase are chemically distinct.

The chemical active may be present in the coating in a concentration from about 0.001 wt % to about 25 wt %, for example.

In some embodiments, the chemical active is electrically conductive. Alternatively, or additionally, the chemical active may be ionically conductive.

In some embodiments, the chemical active is characterized by a diffusivity from about 10⁻¹⁸ m²/s to about 10⁻⁹ m²/s measured at 25° C.

In preferred embodiments, the chemical active is a liquid or is dissolved in a solvent. In other embodiments, the chemical active is a solid. In certain embodiments, the chemical active includes a vapor in combination with a liquid and/or a solid.

The chemical active may be selected from the group consisting of a salt, an acid, a base, an oxidizer, an ionizable compound, an ionic liquid, and combinations thereof. Exemplary oxidizers include, but are not limited to, sodium hypochlorite, hypochlorous acid, and hydrogen peroxide. In certain embodiments, the chemical active is a quaternary ammonium salt.

The chemical active may be an antimicrobial agent, an anticorrosion agent, or a coating protection agent, for example. The chemical active may provide one or more functions to the coating, or may signal when the coating has experienced certain events. For example, in some embodiments, the chemical active is a salt that is sensitive to environmental degradation and can reveal when the coating has undergone ultraviolet (UV) or thermal damage.

In some systems, the first electrode and the second electrode are each pressed against an external surface of the coating. There is preferably a region of coating interposed between the first electrode and the second electrode.

In some systems, one of the first electrode and the second electrode is disposed at or near an external surface of the coating, and the other of the first electrode and the second electrode is distally disposed at the opposite side of the coating.

In some systems, one of the first electrode and the second electrode is pressed against an external surface of the coating, and the other of the first electrode and the second electrode is at least partially embedded within the coating.

In some systems, the first electrode and the second electrode are each at least partially embedded within the coating. In certain systems, the first electrode and the second electrode are each fully embedded within the coating.

In some embodiments, the first electrode and the second electrode are each in the form of concentric circles. In other embodiments, the first electrode and the second electrode are each in the form of parallel lines or parallel shapes.

In some embodiments, the electrical meter is configured to apply an AC waveform to the first electrode and the second electrode. The electrical meter may be configured to sense at a single frequency or at multiple frequencies.

The first electrode, the second electrode, and the electrical meter may be contained in a device (system) that further includes wires, clips, an electricity supply, and a computer, for example.

There is not necessarily a substrate. The principles of the invention may be applied not only to coatings but also to bulk objects.

Some variations provide a system for sensing a chemical active in an object (which may be coated or uncoated), the system comprising:

a chemical active contained within the object, wherein the chemical active is mobile within the object, and wherein the chemical active is ionically and/or electrically conductive;

a first electrode and a second electrode configured to measure AC impedance within the object; and

an electrical meter configured in electrical communication with the first and second electrodes to read a signal corresponding to the AC impedance.

Some variations provide a method of measuring the concentration of a chemical active in a coating, the method comprising:

(a) providing a system comprising: a coating disposed on a substrate; a chemical active contained within the coating, wherein the chemical active is mobile within the coating, and wherein the chemical active is ionically and/or electrically conductive; a first electrode and a second electrode configured to measure AC impedance within the coating; and an electrical meter configured in electrical communication with the first and second electrodes to read a signal corresponding to the AC impedance;

(b) optionally, calibrating the system with known conductivity samples;

(c) optionally, wetting a surface of the coating with a solvent;

(d) pressing the first electrode and the second electrode against the coating;

(e) reading out an impedance value; and

(f) converting the impedance value to a concentration of the chemical active.

In some methods, the chemical active is present in the coating in a concentration from about 0.001 wt % to about 25 wt %.

In some methods, the chemical active is a liquid or is dissolved in a solvent. The chemical active may be selected from the group consisting of a salt, an acid, a base, an oxidizer, an ionizable compound, an ionic liquid, and combinations thereof.

In some methods, step (c) is performed and utilizes an aqueous solvent to wet the surface of the coating.

Some variations provide a method of measuring the concentration of a chemical active in an object, the method comprising:

(a) providing a system comprising: a chemical active contained within the object, wherein the chemical active is mobile within the object, and wherein the chemical active is ionically and/or electrically conductive; a first electrode and a second electrode configured to measure AC impedance within the object; and an electrical meter configured in electrical communication with the first and second electrodes to read a signal corresponding to the AC impedance;

(b) optionally, calibrating the system with known conductivity samples;

(c) optionally, wetting a surface of the object with a solvent;

(d) pressing the first electrode and the second electrode against the object;

(e) reading out an impedance value; and

(f) converting the impedance value to a concentration of the chemical active.

Some variations provide a method of measuring the concentration of a chemical active in a coating, the method comprising:

(a) providing a system comprising: a coating disposed on a substrate; a chemical active contained within the coating, wherein the chemical active is mobile within the coating, and wherein the chemical active is ionically and/or electrically conductive; a first electrode and a second electrode configured to measure AC impedance within the coating; and an electrical meter configured in electrical communication with the first and second electrodes to read a signal corresponding to the AC impedance;

(b) optionally, calibrating the system with known conductivity samples;

(c) adding one or more droplets of a solvent to a surface of the coating;

(d) waiting a selected amount of time;

(e) pressing the first electrode and the second electrode against a surface region comprising the solvent and the coating;

(f) reading out an impedance value; and

(g) converting the impedance value to a concentration of the chemical active.

In some methods, the chemical active is present in the coating in a concentration from about 0.001 wt % to about 25 wt %. The chemical active may be selected from the group consisting of a salt, an acid, a base, an oxidizer, an ionizable compound, an ionic liquid, and combinations thereof.

In some methods, the solvent in step (c) is an aqueous solvent, such as water. The solvent may be a non-aqueous solvent, such as acetone.

In some methods, the selected amount of time in step (d) is from about 10 seconds to about 30 minutes.

Some variations provide a method of measuring the concentration of a chemical active in an object, the method comprising:

(a) providing a system comprising: a chemical active contained within the object, wherein the chemical active is mobile within the object, and wherein the chemical active is ionically and/or electrically conductive; a first electrode and a second electrode configured to measure AC impedance within the object; and an electrical meter configured in electrical communication with the first and second electrodes to read a signal corresponding to the AC impedance;

(b) optionally, calibrating the system with known conductivity samples;

(c) adding one or more droplets of a solvent to a surface of the object;

(d) waiting a selected amount of time;

(e) pressing the first electrode and the second electrode against a surface region comprising the solvent and a portion of the object;

(f) reading out an impedance value; and

(g) converting the impedance value to a concentration of the chemical active.

### BRIEF DESCRIPTION OF THE DRAWINGS

FIG. 1 is an optical microscope image of a biphasic polymer structure containing 25 vol % PEG and 75 vol % pTHF coating, in the Examples.

FIG. 2 is a plot of impedance and conductivity measured as a function of concentration of chemical active (alkyldimethylbenzylammonium chloride) in the coating of the Examples.

FIG. 3 is an exemplary system for sensing a chemical active in a coating disposed on a substrate, in which there is a first electrode and a second electrode each pressed against the external surface of the coating.

FIG. 4 is an exemplary system for sensing a chemical active in a coating disposed on a substrate, in which a first electrode is partially embedded into the coating, and a second electrode is pressed against the external surface of the coating.

FIG. 5 is an exemplary system for sensing a chemical active in a coating disposed on a substrate, in which a first electrode and a second electrode are each partially embedded into the coating.

FIG. 6 is an exemplary system for sensing a chemical active in a coating disposed on a substrate, in which a first electrode is fully embedded into the coating, and a second electrode is fully embedded into the coating.

FIG. 7 is an exemplary system for sensing a chemical active in a coating disposed on a substrate, in which a first electrode is pressed against the surface of the coating, and a second electrode is fully embedded into the coating.

FIG. 8 is an exemplary system for sensing a chemical active in a coating that is not disposed on a substrate, in which a first electrode is pressed against the surface of the coating, and a second electrode is pressed against the opposite surface of the coating.

FIG. 9 is an exemplary system for sensing a chemical active in a coating disposed on a substrate, in which a first electrode is pressed against the surface of the coating, and a coating substrate functions as a second electrode.

FIG. 10 an exemplary method flowchart in some embodiments, for measuring the concentration of a chemical active in a coating, and replenishing the coating with the chemical active, if necessary or desired. Dashed lines denote optional steps.

FIG. 11 is an exemplary method flowchart in some embodiments, for measuring the concentration of a chemical active in a coating, and replenishing the coating with the chemical active, if necessary or desired. Dashed lines denote optional steps.

FIG. 12 is an exemplary method flowchart in some embodiments, for measuring the concentration of a chemical active in a coating, and replenishing the coating with the chemical active, if necessary or desired. Dashed lines denote optional steps.

### DETAILED DESCRIPTION OF EMBODIMENTS OF THE INVENTION

The systems (synonymously, devices) and methods of the present invention will be described in detail by reference to various non-limiting embodiments.

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with the accompanying drawings.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

Unless otherwise indicated, all properties cited in this specification are measured at a temperature of 25° C. and a pressure of 100 kPa (1 bar).

Unless otherwise indicated, all references to “phases” in this patent application are in reference to solid phases or fluid phases. A “phase” is a region of space (forming a thermodynamic system), throughout which all physical properties of a material are essentially uniform. A solid phase is a region of solid material that is chemically uniform and physically distinct from other regions of solid material (or any liquid or vapor materials that may be present). Reference to multiple solid phases in a composition or microstructure means that there are at least two distinct material phases that are solid, without forming a solid solution or homogeneous mixture.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phrase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phrase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms, except when used in a Markush group. Thus in some embodiments not otherwise explicitly recited, any instance of “comprising” may be replaced by “consisting of” or, alternatively, by “consisting essentially of.”

Some variations of the invention provide an impedance sensor that senses the presence of a chemical active in a coating and indicates if the coating needs to be recharged with the chemical active. In this disclosure, “impedance” refers to electrical impedance, and a “chemical active” refers to a chemical species that has at least one activity, i.e. it is not inert.

Impedance sensing allows measuring the chemical active when the coating is in use, without removing anything from the coating or taking a sample to an off-site laboratory. The impedance sensing is enabled by biphasic coatings or polymers with a continuous transport phase that (a) enables rapid and long-range diffusion (on the length scale of millimeters) of chemical actives and (b) has an impedance that depends on the chemical-active concentration.

Conventional coatings or polymers without a fast transport phase have such slow diffusion for chemical actives that it is not possible to sense differences in concentration—all impedances are very high, and small differences are not easily distinguished.

By contrast, the disclosed systems and methods may be configured to return an impedance value in seconds, permitting the coatings to be quickly refilled with chemical actives. The disclosed systems and methods provide a real-time indication of when a function, such as antimicrobial activity or chemical self-decontamination, is not functioning as expected or desired.

Some variations of the invention provide a system for sensing a chemical active in a coating, the system comprising:

a coating disposed on a substrate;

a chemical active contained within the coating, wherein the chemical active is mobile within the coating, and wherein the chemical active is ionically and/or electrically conductive;

a first electrode and a second electrode configured to measure AC impedance within the coating; and

an electrical meter configured in electrical communication with the first and second electrodes to read a signal corresponding to the AC impedance.

The coating may be fabricated from a coating material selected from the group consisting of a polymer, a metal, a ceramic, a preceramic polymer, carbon, and combinations thereof.

In some embodiments, the coating is or includes a polymer. Exemplary polymers include, but are by no means limited to, poly(ethylene glycol), polycarbonate, poly(tetrahydrofuran), and polyurethanes. The polymer may be biphasic, which means the polymer contains at least a first phase that is continuous and a second phase that is discrete or continuous. The first phase and the second phase may be phase-separated on an average length scale of phase separation selected from about 10 nanometers to about 1 millimeter, such as from about 100 nanometers to about 25 microns. Typically, in a biphasic polymer, the first phase and the second phase are chemically distinct. In some embodiments, the two polymer phases of a biphasic polymer are crosslinked together. An exemplary biphasic polymer is one containing a continuous poly(ethylene glycol) phase and a discrete poly(tetrahydrofuran) phase, as described in Examples herein.

The coating or object described herein may be fabricated from polymers described in commonly owned U.S. Pat. No. 10,400,136, issued on Sep. 3, 2019; U.S. Pat. No. 10,619,057, issued on Apr. 14, 2020; and U.S. Patent App. Pub. No. 2021/0386059, published on Dec. 16, 2021, which are hereby incorporated by reference herein.

In some embodiments, the polymer is or includes a polymer selected from the group consisting of a non-fluorinated carbon-based polymer, a silicone, a fluorinated polymer, and combinations thereof. These types of polymers may be preferred when anti-wetting properties (from water or other hydrophilic liquids) are desired for a dry-feel surface. A hydrophobic and/or lyophobic material prevents or minimizes soil adhesion and penetration of debris into the overall structure.

A non-fluorinated carbon-based polymer may be selected from the group consisting of polyalkanes, polyurethanes, polyethers, polyureas, polyesters, polycarbonates, and combinations thereof, for example.

A silicone may be selected from the group consisting of polydimethyl siloxane, polytrifluoropropylmethyl siloxane, polyaminopropylmethyl siloxane, polyaminoethylaminopropylmethyl siloxane, polyaminoethylaminoisobutylmethyl siloxane, and combinations thereof, for example.

A fluorinated polymer may be selected from the group consisting of fluorinated polyols, perfluorocarbons, perfluoropolyethers, polyfluoroacrylates, polyfluorosiloxanes, polyvinylidene fluoride, polytrifluoroethylene, and combinations thereof, for example.

In some embodiments, the polymer is or includes a hygroscopic polymer selected from the group consisting of poly(acrylic acid), poly(ethylene glycol), poly(2-hydroxyethyl methacrylate), poly(vinyl imidazole), poly(2-methyl-2-oxazoline), poly(2-ethyl-2-oxazoline), poly(vinylpyrolidone), modified cellulosic polymers, carboxymethyl cellulose, hydroxyethyl cellulose, hydroxypropyl cellulose, methyl cellulose, and combinations thereof, for example.

In some embodiments, the polymer is or includes a hydrophobic, non-lipophobic polymer selected from the group consisting of poly(propylene glycol) (PPG), poly(tetramethylene glycol) (PTMEG, also known as poly(tetrahydrofuran) or polyTHF), polybutadiene, polycarbonate, polycaprolactone, acrylic polyols, and combinations thereof, for example.

In some embodiments, the polymer is or includes a hydrophilic polymer created with ionic charge that may be present within the polymer as pendant or main-chain carboxylate groups, amine groups, sulfate groups, or phosphate groups, for example. In certain embodiments, monomers containing ionic charge are inserted along the polymer backbone.

In some embodiments, the polymer is or includes an electrolyte polymer selected from the group consisting of polyethylene oxide, polypropylene oxide, polycarbonates, polysiloxanes, polyvinylidene difluoride, and combinations thereof, for example.

In some embodiments, a first polymer is crosslinked, via a crosslinking molecule, with a second polymer in a biphasic polymer. The crosslinking is preferably covalent crosslinking, but can also be ionic crosslinking. When the two polymers are covalently crosslinked, an abrasion-resistant structure is established. Additionally, when the polymers crosslinked, the length scales of the different phases can be better controlled, such as to enhance transport rates of the chemical active.

A crosslinking molecule (when present) may include at least one moiety selected from the group consisting of an amine moiety, a hydroxyl moiety, an isocyanate moiety, and a combination thereof, for example. Other crosslinking molecules may be employed.

In some biphasic polymers, the average length scale of phase separation is from about 0.5 microns to about 100 microns. In certain embodiments, the average length scale of phase separation is from about 1 micron to about 50 microns. In various embodiments, the average length scale of phase separation is from 100 nanometers to 500 microns, 100 nanometers to 600 microns, 100 nanometers to 300 microns, 100 nanometers to 200 microns, 100 nanometers to 100 microns, at least 200 nanometers, at least 500 nanometers, at least 1 micron, at least 5 microns, up to 10 microns, up to 50 microns, up to 100 microns, or up to 500 microns. Exemplary average length scales of phase separation are about, at least about, or at most about 0.1, 0.2, 0.3, 0.4, 0.5, 1, 2, 3, 4, 5, 10, 15, 20, 25, 30, 40, 50, 60, 70, 80, 90, 100, 150, 200, 250, 300, 350, 400, 450, or 500 microns, including any intervening range.

In some embodiments, the coating is or includes a ceramic. Exemplary ceramic coating materials include, but are not limited to, silicon oxycarbide (SiOC), silicon carbide (SiC), silicon nitride (Si₃N₄), silicon oxynitride (SiON), silicon carbonitride (SiCN), silicon boronitride (SiBN), silicon boron carbonitride (SiBCN), boron nitride (BN), and combinations thereof.

In some embodiments, the coating is or includes carbon. Exemplary carbon-containing coating materials include, but are not limited to, graphite, graphene, multi-layer graphene, carbon fibers, carbon nanostructures, pyrolytic carbon, carbon-polymer composites, carbon-ceramic composites, and combinations thereof.

The chemical active may be present in the coating in a concentration selected from about 0.001 wt % to about 25 wt %, for example, on the basis of total weight of the coating. In various embodiments, the concentration of chemical active in the coating may be about, at least about, or at most about 0.001, 0.002, 0.005, 0.01, 0.02, 0.05, 0.1, 0.2, 0.3, 0.4, 0.5, 0.6, 0.7, 0.8, 0.9, 1, 1.1, 1.2, 1.5, 2, 2.5, 3, 4, 5, 6, 7, 8, 9, 10, 11, 12, 15, 20, or 25 wt %, including any intervening ranges.

In some embodiments, the chemical active is dissolved in a solvent to form a chemical-active solution. In other embodiments, the chemical active is a liquid and is not dissolved in a solvent. In other embodiments, the chemical active is a solid. In certain embodiments, the chemical active includes a vapor in combination with a liquid and/or a solid.

Even when not dissolved in a solvent, the chemical active may be part of a chemical-active phase that includes one or more additives, one or more carriers that are not solvents, or other species. An additive may be included to adjust the ionic conductivity of the chemical-active phase, for example.

When the chemical active is dissolved in a solvent to form a chemical-active solution, the concentration of chemical active in the chemical-active solution may be selected from about 0.01 wt % to about 99 wt %, such as from about 0.1 wt % to about 10 wt %. In various embodiments, the concentration of chemical active in the solution may be about, at least about, or at most about 0.01, 0.05, 0.1, 0.2, 0.3, 0.4, 0.5, 0.6, 0.7, 0.8, 0.9, 1, 1.1, 1.2, 1.5, 2, 2.5, 3, 4, 5, 6, 7, 8, 9, 10, 15, 20, 25, 30, 40, 50, 60, 70, 80, 90, 95, 98, 99, 99.5, or 99.9 wt %, including any intervening ranges.

In order to be sensed via impedance, the chemical active is selected to be responsive to an electric field imposed by electrodes. When the chemical active is dissolved in a solvent to form a chemical-active solution, the chemical-active solution is selected to be responsive to an electric field imposed by electrodes. To be responsive to an electric field, the chemical active needs to be ionically conductive, electrically conductive, or both of these. This invention utilizes a measurement of the conductivity of a chemical active contained within a coating, rather than the conductivity of the coating material (e.g., polymer backbone) itself.

The chemical active is preferably ionically conductive. Ionic conductivity refers to the transport of ions, such as ionic species that form the chemical active, or cations and anions that collectively form the chemical active. The chemical active may be characterized by an ionic conductivity selected from about 10⁻⁵ mS/cm to about 10⁻¹ mS/cm, measured at 25° C., for example. In various embodiments, the chemical active is characterized by an ionic conductivity of about, at least about, or at most about 10⁻⁶ mS/cm, 10⁻⁵ mS/cm, 10⁻⁴ mS/cm, 10⁻³ mS/cm, 10⁻² mS/cm, 10⁻¹ mS/cm, or 1 mS/cm, including any intervening range.

If the chemical active is dissolved in a solvent to form a chemical-active solution, the chemical-active solution is preferably ionically conductive. The chemical-active solution may be characterized by an ionic conductivity selected from about 10⁻⁵ mS/cm to about 10⁻¹ mS/cm, measured at 25° C., for example. In various embodiments, the chemical-active solution is characterized by an ionic conductivity of about, at least about, or at most about 10⁻⁶ mS/cm, 10⁻⁵ mS/cm, 10⁻⁴ mS/cm, 10⁻³ mS/cm, 10⁻² mS/cm, 10⁻¹ mS/cm, or 1 mS/cm, including any intervening range

Alternatively, or additionally, the chemical active is electrically conductive. Electrical conductivity refers to the transport of electrons. The transport of electrons may be via ion conduction. For example, an electron attached to a species to form an anion is itself transported along with the anion. Or, electrons may be transported by a charge-transfer mechanism through a solution of ions, in which case electron transport may be faster than ion transport. There may also be direct electron conduction through a neutral form of the chemical active.

The chemical active may be characterized by an electrical conductivity selected from about 10⁻⁵ mS/cm to about 10⁻¹ mS/cm, measured at 25° C., for example. In various embodiments, the chemical active is characterized by an electrical conductivity of about, at least about, or at most about 10⁻⁵ mS/cm, 10⁻⁴ mS/cm, 10⁻³ MS/CM, 10⁻² MS/CM, 10⁻¹ MS/CM, 1 mS/CM, 10 mS/cm, or 100 mS/cm, including any intervening range.

If the chemical active is dissolved in a solvent to form a chemical-active solution, the chemical-active solution is preferably electrically conductive. The chemical-active solution may be characterized by an electrical conductivity selected from about 10⁻⁵ mS/cm to about 10⁻¹ mS/cm, measured at 25° C., for example. In various embodiments, the chemical-active solution is characterized by an electrical conductivity of about, at least about, or at most about 10⁻⁵ mS/cm, 10⁻⁴ mS/cm, 10⁻³ mS/cm, 10⁻² mS/cm, 10⁻¹ mS/cm, 1 mS/cm, 10 mS/cm, or 100 mS/cm, including any intervening range.

In some embodiments, the chemical active is characterized by a diffusivity from about 10⁻¹⁸ m²/s to about 10⁻⁹ m²/s, measured at 25° C., for example. The diffusivity is also referred to as the diffusion coefficient or diffusion constant, and is governed by Fick's law. In various embodiments, the chemical active is characterized by a diffusivity of about, at least about, or at most about 10⁻¹⁸ m²/s, 10⁻¹⁷ m²/s, 10⁻¹⁶ m²/s, 10⁻¹⁵ m²/s, 10⁻¹⁴ m²/s, 10⁻¹³ m²/s, 10⁻¹² m²/s, 10⁻¹¹ m²/s, 10⁻¹⁰ m²/s, or 10⁻⁹ m²/s, including any intervening range. A solvent may enhance the diffusivity of the chemical active, although that is not necessarily the case. The diffusivity is a composite average to account for ion-pair diffusion, free-ion diffusion, and/or other mechanisms of diffusion of the chemical active.

The chemical active preferably does not react with the coating, although a small amount of reversible reactivity may occur. In some embodiments, the chemical active is not covalently bonded to the coating material. In some embodiments, the chemical active in chemically inert with respect to the coating material at 25° C. and 1 bar. “Chemically inert” means that the chemical active and the coating material do not undergo a chemical reaction. Physical forces may exist, such as capillary forces and adsorptive forces, between the chemical active and the coating material.

The chemical active may be an antimicrobial agent, an anticorrosion agent, or a coating protection agent, for example. A coating protection agent may provide biological protection, chemical protection, physical protection, electrical protection, electrochemical protection, magnetic protection, or another type of protection. In certain embodiments, the coating is a self-decontaminating coating that relies on the chemical active for its mechanism of action.

The chemical active may provide one function, or multiple functions (such as 2, 3, 4, or more) to the coating. In certain embodiments, there are multiple coatings, and each coating may contain a distinct chemical active or multiple chemical actives.

As intended in this patent application, “antimicrobial agents” or synonymously “antimicrobial actives” include germicides, bactericides, virucides (antivirals), antifungals, antiprotozoals, antiparasites, and biocides. In some embodiments, antimicrobial agents are specifically bactericides, such as disinfectants, antiseptics, and/or antibiotics. In some embodiments, antimicrobial agents are specifically virucides, or include virucides.

There are many commercial applications of antimicrobial surfaces in homes (e.g., kitchens and bathrooms), in restaurants, on clothing and personal protective equipment, in cars (especially shared-ride vehicles to inhibit the transfer of microbes from one person to another), in airplanes (e.g., for contaminated surfaces that UV light cannot reach), and inside and outside vehicles used to rescue or move people who have been exposed to diseases and pandemics.

The chemical active may be selected from the group consisting of a salt, an acid, a base, an oxidizer, an ionizable compound, an ionic liquid, and combinations thereof. An ionizable compound contains an ionic bond or has a counterion. Exemplary oxidizers include, but are not limited to, sodium hypochlorite, hypochlorous acid, and hydrogen peroxide. In certain embodiments, the chemical active is a quaternary ammonium salt.

In some embodiments, the chemical active is selected from quaternary ammonium molecules. Exemplary quaternary ammonium molecules include, but are not limited to, benzalkonium chloride, benzethonium chloride, methylbenzethonium chloride, cetalkonium chloride, cetylpyridinium chloride, cetrimonium, cetrimide, tetraethylammonium bromide, didecyldimethylammonium chloride, dioctyldimethylammonium chloride, and domiphen bromide. Quaternary ammonium molecules or eutectic mixtures of quaternary ammonium molecules that are liquids at room temperature—ionic liquids or ionic liquid eutectics, respectively—enable fast transport with negligible vapor pressure. A specific example is tetrabutylammonium heptadecafluorooctanesulfonate (C₂₄H₃₆F₁₇NO₃S), which has a melting point less than 5° C. Another specific example is tetraoctylammonium chloride (C₃₂H₆₈ClN) with a melting point of 50-54° C. mixed with tetraheptylammonium chloride (C₂₈H₆₀ClN) with a melting point of 38-41° C. in a eutectic composition ratio that forms a liquid at room temperature (25° C.). Quaternary ammonium molecules may be mixed with imidazolium-based ionic liquids, pyridinium-based ionic liquids, pyrrolidinium-based ionic liquids, and/or phosphonium-based ionic liquids.

In certain embodiments, the chemical active is a salt of a transition metal (e.g., V, Ti, Cr, Co, Ni, Cu, Zn, Tb, W, Ag, Cd, or Au), a salts of a metalloid (e.g., Al, Ga, Ge, As, Se, Sn, Sb, Te, or Bi), a salts of an alkali metal (e.g., Li, Na, or K), a salt of an alkaline earth metal (e.g., Mg or Ca), or a combination thereof.

When the chemical active is a salt, there will be a cation and anion forming the salt. The cation element may be Li, Na, K, Mg, and/or Ca, for example. The anion element or group may be F, Cl, Br, I, SO₃, SO₄, NO₂, NO₃, CH₃COO, and/or CO₃, for example.

In certain embodiments, the chemical active is a water-soluble salt. Exemplary water-soluble salts include, but are not limited to, copper chloride, copper nitrate, zinc chloride, zinc nitrate, silver chloride, silver nitrate, or combinations thereof. Other exemplary water-soluble salts include quaternary ammonium salts, such as (but not limited to) quaternary ammonium molecules.

In certain embodiments, the chemical active is a eutectic liquid salt, which is optionally derived from ammonium salts. The eutectic liquid salt may be antimicrobially active or may provide anticorrosion activity, for example.

In some embodiments, the chemical active is an acid selected from the group consisting of citric acid, lactic acid, phosphoric acid, hydrochloric acid, and combinations thereof.

In some embodiments, the chemical active is a base selected from the group consisting of ammonia, sodium hydroxide, potassium hydroxide, sodium hypochlorite, and combinations thereof.

In some embodiments, the chemical active is selected from oxidizing molecules, such as (but not limited to) those selected from the group consisting of sodium hypochlorite, hypochlorous acid, hydrogen peroxide, and combinations thereof.

In some embodiments, the chemical active is selected from metal ions, such as (but not limited to) silver, copper, zinc, cobalt, nickel, or combinations thereof. Any metal ion with at least some desired activity itself, or which confers a desired activity to a compound to which the metal ion binds, may be employed. The metal ion may be present in a metal complex or a metal salt, for example. In certain embodiments, the chemical active contains a neutral metal (e.g., zero-valent silver, copper, or zinc) which may be dissolved in a liquid and/or may be present as nanoparticles, for example.

When a solvent is utilized, the solvent may be water, a protic inorganic solvent, an aprotic inorganic solvent, a protic organic solvent, an aprotic organic solvent, or a combination thereof. The solvent may be an aqueous solvent or a non-aqueous solvent. The solvent may be polar or non-polar. Exemplary solvents include, but are not limited to, water, ethanol, butanol (any isomer), acetone, acetic acid, methyl acetate, ethyl acetate, ethylene glycol, lactic acid, ethyl lactate, ethylene carbonate, γ-butyrolactone, 2-phenoxyethanol, tetrahydrofuran, and combinations thereof. In some embodiments, the solvent is or includes water that is passively incorporated from atmospheric humidity.

One or more additives may be present with the chemical active, in a chemical-active solution, or in another phase that forms a suspension with the chemical active. Exemplary additives include buffers, UV stabilizers, and particulate fillers.

When an additive is a buffer, it may be an inorganic or organic molecule that maintains a pH value or pH range via acid-base reactions. A buffer may be discrete or may be bonded to the coating material, for example.

When an additive is a UV stabilizer, it may be an antioxidant (e.g., a thiol), a hindered amine (e.g., a derivative of tetramethylpiperidine), UV-absorbing nanoparticles (e.g., TiO₂, ZnO, CdS, CdTe, or ZnS—Ag nanoparticles), or a combination thereof, for example.

When an additive is a particulate filler, it may be selected from the group consisting of silica, alumina, silicates, talc, aluminosilicates, barium sulfate, mica, diatomite, calcium carbonate, calcium sulfate, carbon, wollastonite, and a combination thereof, for example. A particulate filler is optionally surface-modified with a compound selected from the group consisting of fatty acids, silanes, alkylsilanes, fluoroalkylsilanes, silicones, alkyl phosphonates, alkyl phosphonic acids, alkyl carboxylates, alkyldisilazanes, and combinations thereof, for example.

Other additives may be introduced, if desired, to adjust pH, stability, density, viscosity, color, or other properties, for functional, ornamental, safety, or other reasons.

In some embodiments, a chemical active signals when a coating has experienced certain events. For example, the chemical active may be a salt that is sensitive to environmental degradation and can reveal when the coating has undergone UV or thermal damage. For instance, a reduction in the concentration of salt (chemical active) may be a proxy for coating damage. In certain embodiments, the salt is a chemical-active precursor that upon degradation due to sunlight, heat, humidity, or other factors, forms an ionic species that itself is a chemical active that can be detected.

The concentration of the chemical active is measured using electrical impedance. Electrical impedance is a measure of the total opposition that a circuit presents to electric current. Impedance includes both resistance and reactance. The resistance arises from collisions of the current-carrying charged particles with the internal structure of the conductor. The reactance is an additional opposition to the movement of electric charge that arises from the changing magnetic and electric fields in circuits carrying alternating current. The reactance includes both inductive reactance and capacitive reactance. The magnitude of the impedance of a circuit is equal to the maximum value of the potential difference, or voltage, across the circuit, divided by the maximum value of the current (amperes, A) through the circuit. The unit of impedance, like that of resistance, is the ohm (Ω).

The electrical impedance may be determined by applying a small alternating voltage signal to the system. By recording the resulting current, the impedance is calculated by dividing the applied voltage by the measured current. Equivalently, the electrical impedance may be determined by applying a small alternating current signal to the system, and recording the resulting voltage; the impedance is calculated by dividing the measured voltage by the applied current. An impedance spectrum may be recorded by determining impedance at different voltage frequencies, in a frequency sweep, which may be referred to as electrochemical impedance spectroscopy. The present invention does not require a frequency sweep; sensing may be accomplished using a single frequency.

An alternating current or voltage is applied across electrodes—in particular, a first electrode and a second electrode. Because alternating current and voltage are utilized, the first electrode frequently switches between being a positive electrode and a negative electrode, and conversely, the second electrode frequently switches between being a negative electrode and a positive electrode. At a given (arbitrary) point in time, there is at least one positive electrode and at least one negative electrode.

In some embodiments, the device has pins that penetrate a certain distance into the film. One pin may be a long electrode that penetrates to the underlying substrate, partially embedding the long electrode. The other pin may be a short electrode that contacts the surface. Electrodes may be fully embedded, with electrical leads (e.g., metal wires) to the meter. Additional electrodes, such as a reference electrode or a spare electrode, may also be present.

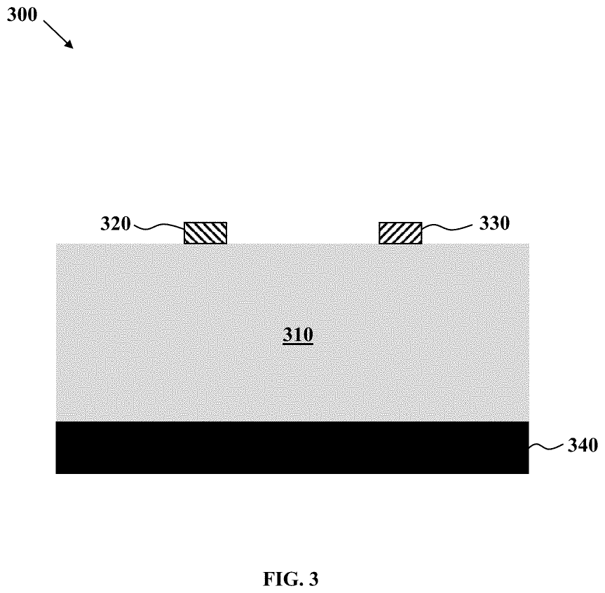

In some embodiments, the first electrode and the second electrode are each in the form of concentric circles. In other embodiments, the first electrode and the second electrode are each in the form of parallel lines or parallel shapes. Parallel electrodes are depicted in FIGS. 3 to 9, but it should be understood that the present invention is not limited to parallel electrodes.

In some systems, the first electrode and the second electrode are each pressed against an external surface of the coating. There is preferably a region of coating interposed between the first electrode and the second electrode. See, for example, FIG. 3. FIG. 3 is an exemplary system 300 for sensing a chemical active (not shown) in a coating 310 disposed on a substrate 340. A first electrode 320 and a second electrode 330 are spaced apart and are each pressed against the external surface of the coating 310. The substrate is optional in FIG. 3 and in all drawings herein. Also note that FIG. 3 is a two-dimensional side view or top view of a three-dimensional structure, and is not drawn to scale.

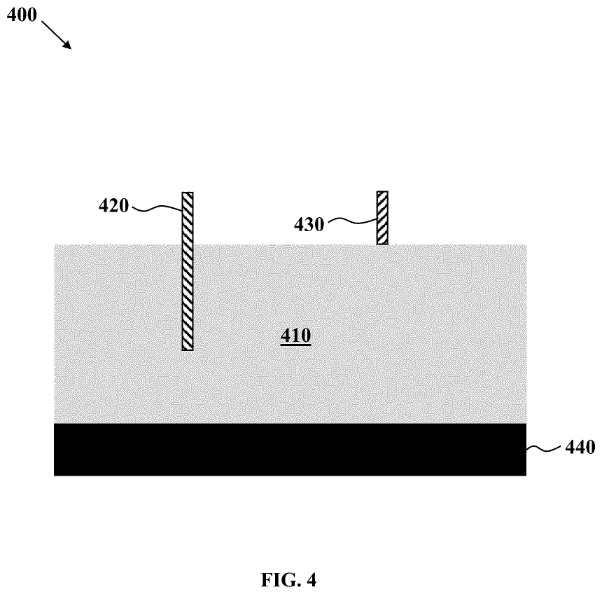

In some systems, one of the first electrode and the second electrode is pressed against an external surface of the coating, and the other of the first electrode and the second electrode is at least partially embedded within the coating. See, for example, FIG. 4. FIG. 4 is an exemplary system 400 for sensing a chemical active (not shown) in a coating 410 disposed on a substrate 440. A first electrode 420 is partially embedded into the coating 410, and a second electrode 430 is pressed against the external surface of the coating 410. The first electrode 420 and second electrode 430 are spaced apart.

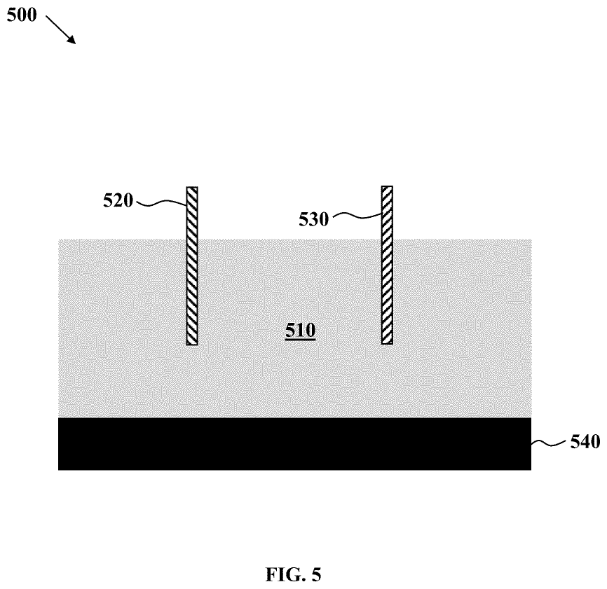

In some systems, the first electrode and the second electrode are each at least partially embedded within the coating. See, for example, FIG. 5. FIG. 5 is an exemplary system 500 for sensing a chemical active (not shown) in a coating 510 disposed on a substrate 540. A first electrode 520 is partially embedded into the coating 510, and a second electrode 530 is partially embedded into the coating 510. The first electrode 520 and second electrode 530 are spaced apart.

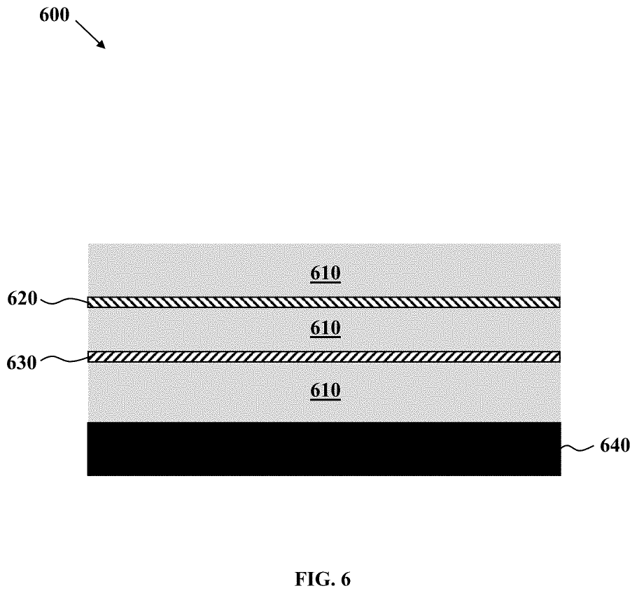

In certain systems, the first electrode and the second electrode are each fully embedded within the coating. See, for example, FIG. 6. FIG. 6 is an exemplary system 600 for sensing a chemical active (not shown) in a coating 610 disposed on a substrate 640. A first electrode 620 is fully embedded into the coating 610, and a second electrode 630 is fully embedded into the coating 610. The first electrode 620 and second electrode 630 are spaced apart in the direction from the substrate to the coating surface, which forms a coating 610 region between electrodes 620, 630 in addition to coating 610 regions above the first electrode 620 and below the second electrode 630. The specific positions of the electrodes 620, 630 may vary; for example, the second electrode 630 may be adjacent to the substrate 640, the first electrode 620 may be disposed at or near the coating 610 surface, and/or the electrodes 620, 630 may be switched.

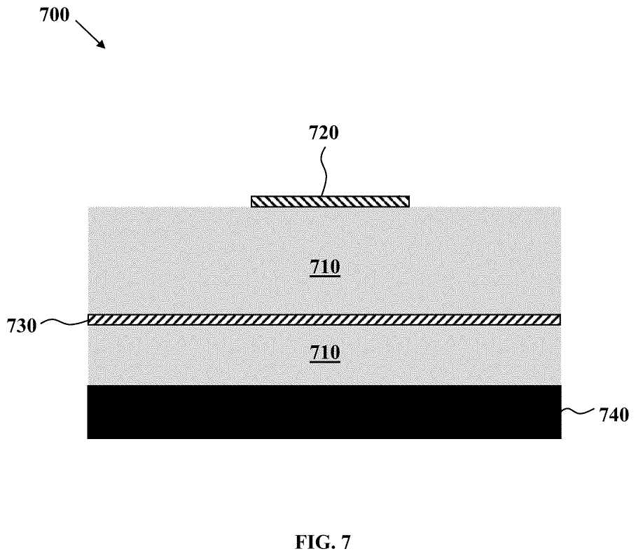

In some systems, one of the first electrode and the second electrode is disposed at or near an external surface of the coating, and the other of the first electrode and the second electrode is fully embedded within the coating. See, for example, FIG. 7. FIG. 7 is an exemplary system 700 for sensing a chemical active (not shown) in a coating 710 disposed on a substrate 740. A first electrode 720 is pressed against the surface of the coating 710, and a second electrode 730 is fully embedded into the coating 710. The first electrode 720 and second electrode 730 are spaced apart in the direction from the substrate to the coating surface, which forms a coating 710 region between electrodes 720, 730 in addition to a coating 710 between the second electrode 730 and the substrate 740.

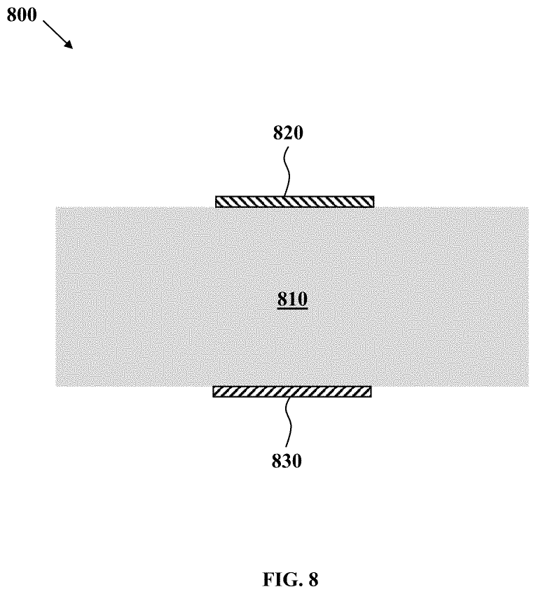

In some systems, one of the first electrode and the second electrode is disposed at or near an external surface of the coating, and the other of the first electrode and the second electrode is distally disposed at the opposite side of the coating. See, for example, FIG. 8. FIG. 8 is an exemplary system 800 for sensing a chemical active (not shown) in a coating 810 that is not disposed on a substrate. A first electrode 820 is pressed against the surface of the coating 810, and a second electrode 830 is pressed against the opposite surface of the coating 810. The first electrode 820 and second electrode 830 are spaced apart in the direction between coating surfaces.

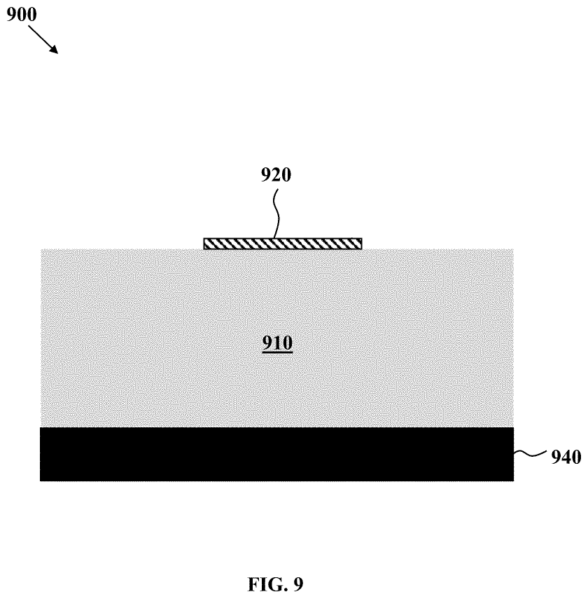

In some systems, a coating substrate itself functions as an electrode. See, for example, FIG. 9. FIG. 9 is an exemplary system 900 for sensing a chemical active (not shown) in a coating 910 disposed on a substrate 940. A first electrode 920 is pressed against the surface of the coating 910. The coating 910 is disposed on a coating substrate 940. The coating substrate 940 functions as a second electrode and is spaced apart in the direction from the substrate to the coating surface, which forms a coating 910 region between electrodes.

In some embodiments, the electrical meter is configured to apply an alternating-current (AC) waveform to the first electrode and the second electrode. An AC waveform, also known as an AC sinusoidal waveform, is created by rotating a coil within a magnetic field. The electrical meter measures the electrical impedance across the electrodes. The electrical meter may be configured to sense at a single AC frequency or at multiple AC frequencies. In some embodiments, the electrical meter collects an impendence versus AC frequency spectrum and fits the data for impedance. If an AC frequency sweep is used, the meter may also perform a fit to the impedance data.

The electrical meter may contain a calibration table, or an equation, to convert the measured impedance to the concentration of the chemical active. The calibration table (e.g., a look-up table) or equation (e.g., correlation formula) may be obtained from previous measurements using known chemical-active concentrations, previous experiments, computer-assisted simulations, theoretical principles, or a combination thereof.

Some embodiments are designed to provide quantitative sensing, to determine an exact value of the chemical-active concentration, within an acceptable tolerance for accuracy. Other embodiments are intended to provide qualitative sensing of a chemical active. In qualitative sensing, the exact chemical-active concentration may be less relevant than the derivative of the concentration with time or with space. In certain embodiments, the mere fact that the concentration of chemical active is decreasing, or has decreased, is sufficient to take action—e.g., a step to replenish the coating with more chemical active.

The first electrode, the second electrode, and the electrical meter may be contained in a device (synonymously, a system) that further includes metal wires, an electricity supply, clips, and a computer, for example. Metal wires may serve as electrical leads or current collectors to an electrode. The metal wires may be made from any suitable materials, such as (but not limited to) Al, Cu, Ni, Ti, Au, or Pt. An electricity supply provides an external circuit through which current passes. Clips may be used to hold wires to surfaces, to hold electrodes, to make an electrical connection with the electrical meter, and so on.

A “computer” utilized in the system is any programmable computing device, or plurality of devices which may be distributed in time or space, capable of being programmed (such as using C++ programming language) or otherwise caused to execute code for executing one or more steps of a method described herein. The algorithm may be embedded within a controller. In some embodiments, the computer has a processor, an area of main memory for executing program code under the direction of the processor, a storage device for storing data and program code and a bus connecting the processor, main memory, and the storage device; the code being stored in the storage device and executing in the main non-transient memory under the direction of the processor, to perform one or more steps of a method recited in this description. Optionally, the computer may be configured to exchange data with a network (such as the Internet), and may carry out calculations on remote computers or servers, or via cloud computing.

A computer system may be configured to perform calculations, processes, operations, and/or functions associated with a program or algorithm. In some embodiments, certain steps discussed herein are realized as a series of instructions (e.g., software program) that reside within computer-readable memory units and are executed by one or more processors of a computer system. When executed, the instructions cause the computer system to perform specific actions and exhibit specific behavior, such as described herein. Examples of steps that may be performed by a computer include generating an AC waveform, calculating an impedance spectrum, and correlating impedance to a concentration of chemical active.

A computer system may include an address/data bus that is configured to communicate information. Additionally, one or more data processing units are coupled with an address/data bus. A processor is configured to process information and instructions. In some embodiments, a processor is a microprocessor. Alternatively, a processor may be a different type of processor such as a parallel processor, or a field-programmable gate array. A computer system may be configured to utilize one or more data-storage units. A computer system may include a volatile memory unit, such as (but not limited to) random access memory (“RAM”), static RAM, or dynamic RAM, coupled with an address/data bus, wherein a volatile memory unit is configured to store information and instructions for a processor. A computer system further may include a non-volatile memory unit, such as (but not limited to) read-only memory (“ROM”), programmable ROM (“PROM”), erasable programmable ROM (“EPROM”), electrically erasable programmable ROM (“EEPROM”), or flash memory coupled with an address/data bus, wherein non-volatile memory unit is configured to store static information and instructions for a processor. The computer system may execute instructions retrieved from an online data-storage unit such as in cloud computing.

In some embodiments, the computer system also may include one or more interfaces coupled with the address/data bus. The communication interfaces implemented by the one or more interfaces may include wireline (e.g., serial cables, modems, network adaptors, etc.) and/or wireless (e.g., wireless modems, wireless network adaptors, etc.) communication technology. In some embodiments, a display device is coupled with an address/data bus, wherein the display device is configured to display video and/or graphics. A display device may include a cathode ray tube (“CRT”), liquid crystal display (“LCD”), field emission display (“FED”), plasma display or any other display device suitable for displaying video and/or graphic images and alphanumeric characters recognizable to a user.

In some embodiments, the computer system may include an input device coupled with the address/data bus, wherein the input device is configured to communicate information and command selections to a processor. The input device may be an alphanumeric input device, such as a keyboard. In some embodiments, the computer system may include a cursor control device coupled with the address/data bus, wherein the cursor control device is configured to communicate user input information and/or command selections to a processor. A cursor control device may be implemented using a device such as a mouse, a track-ball, a track-pad, an optical tracking device, or a touch screen. A cursor control device may alternatively, or additionally, be directed and/or activated via input from input device, such as in response to the use of special keys and key sequence commands associated with an input device. Alternatively, or additionally, the cursor control device may be configured to be directed or guided by voice commands.

The substrate, if present, may vary widely, depending on the particular coating application. The substrate may be metallic, polymeric, carbonaceous, ceramic, glassy, or a combination thereof. Exemplary substrates include aluminum, stainless steel, titanium, alumina, silica, silicon carbide, polycarbonate, polypropylene, polyurethane, poly(vinyl chloride), wood, natural rubber, and combinations thereof.

In some embodiments, an adhesion layer is disposed on a substrate, wherein the adhesion layer is configured to promote adhesion of the coating to the selected substrate. An adhesion layer contains one or more adhesion-promoting materials, such as (but not limited to) primers (e.g., carboxylated styrene-butadiene polymers), alkoxysilanes, zirconates, and titanium alkoxides.

There is not necessarily a substrate. The principles of the invention may be applied not only to coatings but also to bulk objects. That is, the sensing methodology described herein also will work for a region of an object at or near a surface, or for an object that is fabricated entirely from a biphasic-polymer coating material. The object may be essentially a slab of coating thick enough to not need to be mechanically supported by a substrate. Alternatively, an object may have a region near the surface that is deemed to be the coating while the rest of the object is deemed to be the substrate, wherein the coating and substrate may have the same composition or different compositions.

The coating or object may have a thickness from about 1 μm to about 10 mm, for example. In various embodiments, the coating or object thickness is about 100 nm, 1 μm, 10 μm, 100 μm, 1 mm, or 10 mm. Thicker coatings provide the benefit that even after surface abrasion, the coating still functions because the entire depth of the coating (not just the outer surface) contains the functional materials. The coating or object thickness will generally depend on the specific application.

Some variations thus provide a system for sensing a chemical active in an object (which may be coated or uncoated), the system comprising:

a chemical active contained within the object, wherein the chemical active is mobile within the object, and wherein the chemical active is ionically and/or electrically conductive;

a first electrode and a second electrode configured to measure AC impedance within the object; and

an electrical meter configured in electrical communication with the first and second electrodes to read a signal corresponding to the AC impedance.

The object may be fabricated from a coating material selected from the group consisting of a polymer, a metal, a ceramic, carbon, and combinations thereof.

In some embodiments, the object is or includes a polymer. Exemplary polymers include, but are by no means limited to, poly(ethylene glycol), polycarbonate, poly(tetrahydrofuran), and polyurethanes. The polymer may be biphasic, which means the polymer contains at least a first phase that is continuous and a second phase that is discrete or continuous. The first phase and the second phase may be phase-separated on an average length scale of phase separation selected from about 10 nanometers to about 1 millimeter, such as from about 100 nanometers to about 25 microns. Typically, in a biphasic polymer, the first phase and the second phase are chemically distinct. An exemplary biphasic polymer is one containing a continuous poly(ethylene glycol) phase and a discrete poly(tetrahydrofuran) phase, as described in Examples herein.

The chemical active may be present in the object in a concentration selected from about 0.001 wt % to about 25 wt %, for example, on the basis of total weight of the object. In various embodiments, the concentration of chemical active in the object may be about, at least about, or at most about 0.001, 0.002, 0.005, 0.01, 0.02, 0.05, 0.1, 0.2, 0.3, 0.4, 0.5, 0.6, 0.7, 0.8, 0.9, 1, 1.1, 1.2, 1.5, 2, 2.5, 3, 4, 5, 6, 7, 8, 9, 10, 11, 12, 15, 20, or 25 wt %, including any intervening ranges (e.g., 0.1-8 wt %).

The chemical active preferably does not react with the material of the object, although a small amount of reversible reactivity may occur. In some embodiments, the chemical active in chemically inert with respect to the object material at 25° C. and 1 bar. “Chemically inert” means that the chemical active and the object material do not undergo a chemical reaction. Physical forces may exist, such as capillary forces and adsorptive forces, between the chemical active and the object material.

The chemical active may be selected from the group consisting of a salt, an acid, a base, an oxidizer, an ionizable compound, an ionic liquid, and combinations thereof. Exemplary oxidizers include, but are not limited to, sodium hypochlorite, hypochlorous acid, and hydrogen peroxide. In certain embodiments, the chemical active is a quaternary ammonium salt.

The chemical active may be an antimicrobial agent, an anticorrosion agent, or an structural-protection agent, for example. The chemical active may provide one function, or multiple functions (such as 2, 3, 4, or more) to the object.

In some embodiments, a chemical active signals when an object has experienced certain events. For example, the chemical active may be a salt that is sensitive to environmental degradation and can reveal when the object has undergone UV or thermal damage. For instance, a reduction in the concentration of salt (chemical active) may be a proxy for damage to the object. In certain embodiments, the salt is a chemical-active precursor that upon degradation due to sunlight, heat, humidity, or other factors, forms an ionic species that itself is a chemical active that can be detected.

In an object, the concentration of the chemical active is measured using electrical impedance, in the same manner as described above for coatings. The drawings of FIGS. 3 to 9 may depict objects by replacing the term “coating” with “object region” (310, 410, etc.). It is possible for an object to contain a first chemical active, wherein the object is coated with a coating that contains a second chemical active. The substrate (340, 440, etc.) is optional in all objects.

The geometry of the object may vary widely. Examples include flat plates, sheets, panels, bars, rods, beams, curved structures, and arbitrary geometries. The object may be fabricated using known techniques, including additive manufacturing, to create essentially any 3D geometry. Typically, when the object incorporates a coating, the coating takes on the geometry of the object, although the coating thickness may vary along the length of the object and/or there may be coating features built into the object surface, such as pillars pointing out from the surface.

Various features may be incorporated into the object to increase overall efficacy, depending on the intended activity provided by the chemical active. For example, an antimicrobial coating may include physical features (e.g., nanorods or nanoporosity) that is of a similar length scale of viruses (e.g., about 50-150 nanometers) so that such physical features enhance the capture of viruses at the surface of the coating or object. The physical features may be fabricated from tethered quaternary ammonium compounds, alkyl chains, or curable polycations (e.g., polyethyleneimine), for example.

Some variations provide a method of measuring the concentration of a chemical active in a coating, the method comprising:

(a) providing a system comprising: a coating disposed on a substrate; a chemical active contained within the coating, wherein the chemical active is mobile within the coating, and wherein the chemical active is ionically and/or electrically conductive; a first electrode and a second electrode configured to measure AC impedance within the coating; and an electrical meter configured in electrical communication with the first and second electrodes to read a signal corresponding to the AC impedance;

(b) optionally, calibrating the system with known conductivity samples;

(c) optionally, wetting a surface of the coating with a solvent;

(d) pressing the first electrode and the second electrode against the coating;

(e) reading out an impedance value; and

(f) converting the impedance value to a concentration of the chemical active.

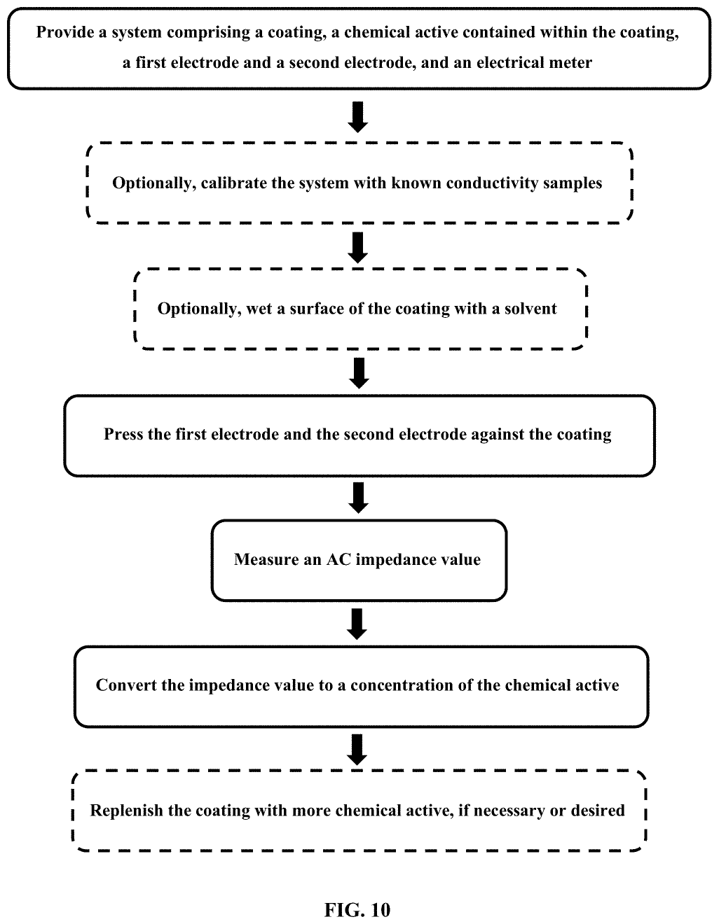

FIG. 10 an exemplary method flowchart in some embodiments, for measuring the concentration of a chemical active in a coating, and replenishing the coating with the chemical active, if necessary or desired. Dashed lines denote optional steps.

In some methods, the chemical active is present in the coating in a concentration from about 0.001 wt % to about 25 wt %, for example, on the basis of total weight of the coating. In various embodiments, the concentration of chemical active in the coating may be about, at least about, or at most about 0.001, 0.002, 0.005, 0.01, 0.02, 0.05, 0.1, 0.2, 0.3, 0.4, 0.5, 0.6, 0.7, 0.8, 0.9, 1, 1.1, 1.2, 1.5, 2, 2.5, 3, 4, 5, 6, 7, 8, 9, 10, 11, 12, 15, 20, or 25 wt %, including any intervening ranges.

In some methods, the chemical active is a liquid or is dissolved in a solvent. In some methods, step (c) is performed and utilizes an aqueous solvent to wet the surface of the coating.

The chemical active may be selected from the group consisting of a salt, an acid, a base, an oxidizer, an ionizable compound, an ionic liquid, and combinations thereof.

In some methods, steps (c), (d), (e), and (f) are collectively performed in a period of time from about 1 second to about 10 minutes, such as from about 10 seconds to about 60 seconds. The ability to return a value (concentration of chemical active) so quickly enables the coating to be refilled with more chemical active almost immediately after its concentration has fallen to an undesirably low level.

Some variations provide a method of measuring the concentration of a chemical active in an object, the method comprising:

(a) providing a system comprising: a chemical active contained within the object, wherein the chemical active is mobile within the object, and wherein the chemical active is ionically and/or electrically conductive; a first electrode and a second electrode configured to measure AC impedance within the object; and an electrical meter configured in electrical communication with the first and second electrodes to read a signal corresponding to the AC impedance;

(b) optionally, calibrating the system with known conductivity samples;

(c) optionally, wetting a surface of the object with a solvent;

(d) pressing the first electrode and the second electrode against the object;

(e) reading out an impedance value; and

(f) converting the impedance value to a concentration of the chemical active.

In some methods, the chemical active is present in the object in a concentration from about 0.001 wt % to about 25 wt %, for example, on the basis of total weight of the object. In various embodiments, the concentration of chemical active in the object may be about, at least about, or at most about 0.001, 0.002, 0.005, 0.01, 0.02, 0.05, 0.1, 0.2, 0.3, 0.4, 0.5, 0.6, 0.7, 0.8, 0.9, 1, 1.1, 1.2, 1.5, 2, 2.5, 3, 4, 5, 6, 7, 8, 9, 10, 11, 12, 15, 20, or 25 wt %, including any intervening ranges.

In some methods applied to an object, the chemical active is a liquid or is dissolved in a solvent. In some methods, step (c) is performed and utilizes an aqueous solvent to wet the surface of the coating.

The chemical active may be selected from the group consisting of a salt, an acid, a base, an oxidizer, an ionizable compound, an ionic liquid, and combinations thereof.

In some methods applied to an object, steps (c), (d), (e), and (f) are collectively performed in a period of time from about 1 second to about 10 minutes, such as from about 10 seconds to about 60 seconds. The ability to return a value (concentration of chemical active) so quickly enables the object to be refilled with more chemical active almost immediately after its concentration has fallen to an undesirably low level.

Some variations provide a method of measuring the concentration of a chemical active in a coating, the method comprising:

(a) providing a system comprising: a coating disposed on a substrate; a chemical active contained within the coating, wherein the chemical active is mobile within the coating, and wherein the chemical active is ionically and/or electrically conductive; a first electrode and a second electrode configured to measure AC impedance within the coating; and an electrical meter configured in electrical communication with the first and second electrodes to read a signal corresponding to the AC impedance;

(b) optionally, calibrating the system with known conductivity samples;

(c) adding one or more droplets of a solvent to a surface of the coating;

(d) waiting a selected amount of time;

(e) pressing the first electrode and the second electrode against a surface region comprising the solvent and the coating;

(f) reading out an impedance value; and

(g) converting the impedance value to a concentration of the chemical active.

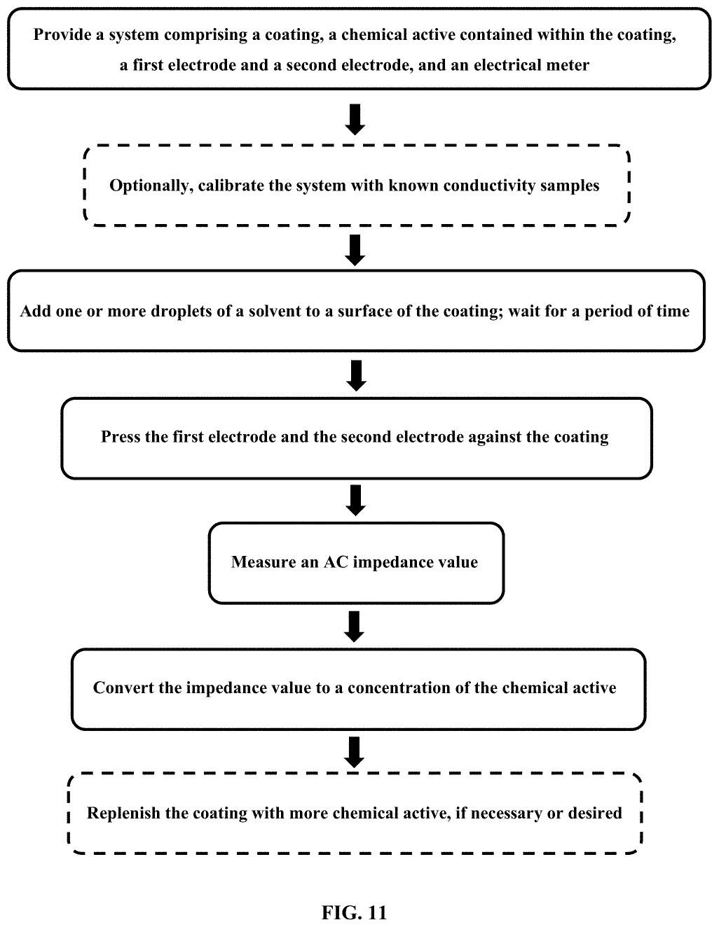

FIG. 11 is an exemplary method flowchart in some embodiments, for measuring the concentration of a chemical active in a coating, and replenishing the coating with the chemical active, if necessary or desired. Dashed lines denote optional steps.

In some methods, the chemical active is present in the coating in a concentration from about 0.001 wt % to about 25 wt %, for example, on the basis of total weight of the coating. In various embodiments, the concentration of chemical active in the coating may be about, at least about, or at most about 0.001, 0.002, 0.005, 0.01, 0.02, 0.05, 0.1, 0.2, 0.3, 0.4, 0.5, 0.6, 0.7, 0.8, 0.9, 1, 1.1, 1.2, 1.5, 2, 2.5, 3, 4, 5, 6, 7, 8, 9, 10, 11, 12, 15, 20, or 25 wt %, including any intervening ranges.

The chemical active may be selected from the group consisting of a salt, an acid, a base, an oxidizer, an ionizable compound, an ionic liquid, and combinations thereof.

In some methods, the solvent in step (c) is an aqueous solvent, such as water. The solvent may be a non-aqueous solvent, such as acetone or ethanol, or a mixture of water and a non-aqueous solvent that is preferably miscible in water.

In some methods, the selected amount of time in step (d) is from about 10 seconds to about 30 minutes, such as from about 30 seconds to about 5 minutes. In various embodiments, the amount of wait time in step (d) is about, at least about, or at most about 10 seconds, 20 seconds, 30 seconds, 60 seconds, 2 minutes, 3 minutes, 4 minutes, 5 minutes, 10 minutes, 15 minutes, 20 minutes, or 30 minutes, including any intervening range.

In some methods employing the addition of droplets of a solvent following by waiting, steps (c), (d), (e), (f), and (g) are collectively performed in a period of time from about 10 seconds to about 15 minutes, such as from about 30 seconds to about 2 minutes. The ability to return a value (concentration of chemical active) so quickly enables the coating to be refilled with more chemical active almost immediately after its concentration has fallen to an undesirably low level.

Some variations provide a method of measuring the concentration of a chemical active in an object, the method comprising:

(a) providing a system comprising: a chemical active contained within the object, wherein the chemical active is mobile within the object, and wherein the chemical active is ionically and/or electrically conductive; a first electrode and a second electrode configured to measure AC impedance within the object; and an electrical meter configured in electrical communication with the first and second electrodes to read a signal corresponding to the AC impedance;

(b) optionally, calibrating the system with known conductivity samples;

(c) adding one or more droplets of a solvent to a surface of the object;

(d) waiting a selected amount of time;

(e) pressing the first electrode and the second electrode against a surface region comprising the solvent and a portion of the object;

(f) reading out an impedance value; and

(g) converting the impedance value to a concentration of the chemical active.

In some methods, the chemical active is present in the object in a concentration from about 0.001 wt % to about 25 wt %, for example, on the basis of total weight of the object. In various embodiments, the concentration of chemical active in the object may be about, at least about, or at most about 0.001, 0.002, 0.005, 0.01, 0.02, 0.05, 0.1, 0.2, 0.3, 0.4, 0.5, 0.6, 0.7, 0.8, 0.9, 1, 1.1, 1.2, 1.5, 2, 2.5, 3, 4, 5, 6, 7, 8, 9, 10, 11, 12, 15, 20, or 25 wt %, including any intervening ranges.

The chemical active may be selected from the group consisting of a salt, an acid, a base, an oxidizer, an ionizable compound, an ionic liquid, and combinations thereof.

In some methods applied to an object, the solvent in step (c) is an aqueous solvent, such as water. The solvent may be a non-aqueous solvent, such as acetone, or a mixture of water and a non-aqueous solvent that is preferably miscible in water.

In some methods applied to an object, the selected amount of time in step (d) is from about 10 seconds to about 30 minutes, such as from about 30 seconds to about 5 minutes. In various embodiments, the amount of wait time in step (d) is about, at least about, or at most about 10 seconds, 20 seconds, 30 seconds, 60 seconds, 2 minutes, 3 minutes, 4 minutes, 5 minutes, 10 minutes, 15 minutes, 20 minutes, or 30 minutes, including any intervening range.

In some methods employing the addition of droplets of a solvent to an object following by waiting, steps (c), (d), (e), (f), and (g) are collectively performed in a period of time from about 10 seconds to about 15 minutes, such as from about 30 seconds to about 2 minutes. The ability to return a value (concentration of chemical active) so quickly enables the object to be refilled with more chemical active almost immediately after its concentration has fallen to an undesirably low level.

Some variations provide a method of measuring the concentration of a chemical active in a coating, the method comprising:

(a) providing a system comprising: a coating disposed on a substrate; a chemical active contained within the coating, wherein the chemical active is mobile within the coating, and wherein the chemical active is ionically and/or electrically conductive; a first electrode and a second electrode configured to measure AC impedance within the coating, wherein at least one of the electrodes is embedded within the coating; and an electrical meter configured in electrical communication with the first and second electrodes to read a signal corresponding to the AC impedance;

(b) optionally, calibrating the system with known conductivity samples;

(c) optionally, wetting a surface of the coating with a solvent;

(d) connecting the first electrode and the second electrode to the electrical meter;

(e) reading out an impedance value; and

(f) converting the impedance value to a concentration of the chemical active.

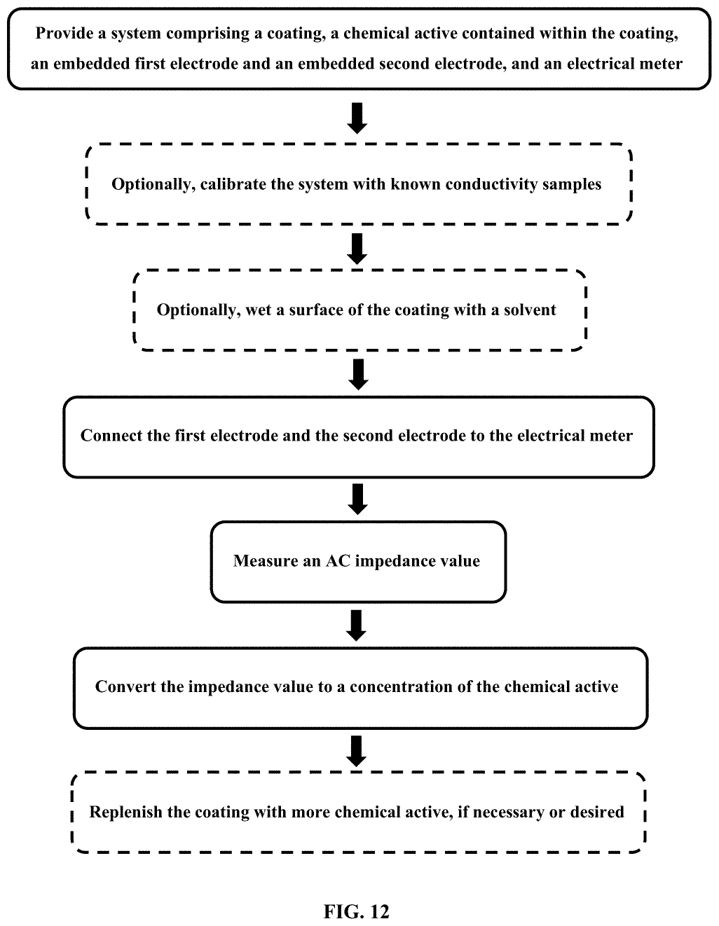

FIG. 12 is an exemplary method flowchart in some embodiments, for measuring the concentration of a chemical active in a coating, and replenishing the coating with the chemical active, if necessary or desired. Dashed lines denote optional steps.

Some variations provide a method of measuring the concentration of a chemical active in an object, the method comprising:

(a) providing a system comprising: a chemical active contained within the object, wherein the chemical active is mobile within the object, and wherein the chemical active is ionically and/or electrically conductive; a first electrode and a second electrode configured to measure AC impedance within the object; and an electrical meter configured in electrical communication with the first and second electrodes to read a signal corresponding to the AC impedance;

(b) optionally, calibrating the system with known conductivity samples;

(c) optionally, wetting a surface of the object with a solvent;

(d) connecting the first electrode and the second electrode to the electrical meter;

(e) reading out an impedance value; and

(f) converting the impedance value to a concentration of the chemical active.

In any of the disclosed methods, an additional step of replenishing the coating or object with more chemical active may be performed, if necessary or desired. This additional step is, strictly speaking, optional since the measurement method might never show the chemical-active concentration to be too low, or the replenishment might be skipped for some reason, such as the coating functionality no longer being needed.

Some embodiments provide a method of replenishing a chemical active in a coating, the method comprising:

(a) providing a system comprising: a coating disposed on a substrate; a chemical active contained within the coating, wherein the chemical active is mobile within the coating, and wherein the chemical active is ionically and/or electrically conductive; a first electrode and a second electrode configured to measure AC impedance within the coating; and an electrical meter configured in electrical communication with the first and second electrodes to read a signal corresponding to the AC impedance;

(b) optionally, calibrating the system with known conductivity samples;

(c) optionally, wetting a surface of the coating;

(d) pressing the first electrode and the second electrode against the coating;

(e) reading out an impedance value;

(f) converting the impedance value to a concentration of the chemical active; and

(g) if the concentration of the chemical active is determined to be too low, replenishing the coating with chemical active.

Some embodiments provide a method of replenishing a chemical active in a coating, the method comprising:

(a) providing a system comprising: a coating disposed on a substrate; a chemical active contained within the coating, wherein the chemical active is mobile within the coating, and wherein the chemical active is ionically and/or electrically conductive; a first electrode and a second electrode configured to measure AC impedance within the coating; and an electrical meter configured in electrical communication with the first and second electrodes to read a signal corresponding to the AC impedance;

(b) optionally, calibrating the system with known conductivity samples;

(c) adding one or more droplets of a solvent to a surface of the coating;

(d) waiting a selected amount of time;

(e) pressing the first electrode and the second electrode against a surface region comprising the solvent and the coating;

(f) reading out an impedance value;

(g) converting the impedance value to a concentration of the chemical active; and

(h) if the concentration of the chemical active is determined to be too low, replenishing the coating with chemical active.

Some embodiments provide a method of replenishing a chemical active in an object, the method comprising:

(a) providing a system comprising: a chemical active contained within the object, wherein the chemical active is mobile within the object, and wherein the chemical active is ionically and/or electrically conductive; a first electrode and a second electrode configured to measure AC impedance within the object; and an electrical meter configured in electrical communication with the first and second electrodes to read a signal corresponding to the AC impedance;

(b) optionally, calibrating the system with known conductivity samples;

(c) optionally, wetting a surface of the object;

(d) pressing the first electrode and the second electrode against the object;

(e) reading out an impedance value;

(f) converting the impedance value to a concentration of the chemical active; and

(g) if the concentration of the chemical active is determined to be too low, replenishing the object with chemical active.

Some embodiments provide a method of replenishing a chemical active in an object, the method comprising:

(a) providing a system comprising: a chemical active contained within the object, wherein the chemical active is mobile within the object, and wherein the chemical active is ionically and/or electrically conductive; a first electrode and a second electrode configured to measure AC impedance within the object; and an electrical meter configured in electrical communication with the first and second electrodes to read a signal corresponding to the AC impedance;

(b) optionally, calibrating the system with known conductivity samples;

(c) adding one or more droplets of a solvent to a surface of the object;

(d) waiting a selected amount of time;

(e) pressing the first electrode and the second electrode against a surface region comprising the solvent and the object;

(f) reading out an impedance value;

(g) converting the impedance value to a concentration of the chemical active; and

(h) if the concentration of the chemical active is determined to be too low, replenishing the object with chemical active.

Replenishing the coating or object with more chemical active may be done continuously or intermittently, depending on the results of the measurements. When the coating or object is to be replenished, the initial concentration of chemical active may be restored, or a different concentration may be used, which may be lower or higher than the original value. For example, if it is found that the chemical active is being depleted relatively quickly, the replenishment concentration may be higher than the original concentration. Other factors may be involved in the desired replenishment concentration, such as environmental changes, time of year, performance, cost, or other reasons. The decision to replenish and the replenishment quantity, if any, may be made automatically, such as in a computer, or may be made manually on a case-by-case basis.

The coating or object containing the chemical active may be used in a variety of temperature ranges. Exemplary temperatures are from about −30° C. to 60° C., such as from about −10° C. to about 50° C.

### Examples

These Examples describe impedance sensing of a chemical active within a biphasic polymer coating. The chemical active is alkyldimethylbenzylammonium chloride and the biphasic polymer is poly(ethylene glycol)/poly(tetrahydrofuran).

### Fabrication of Coating

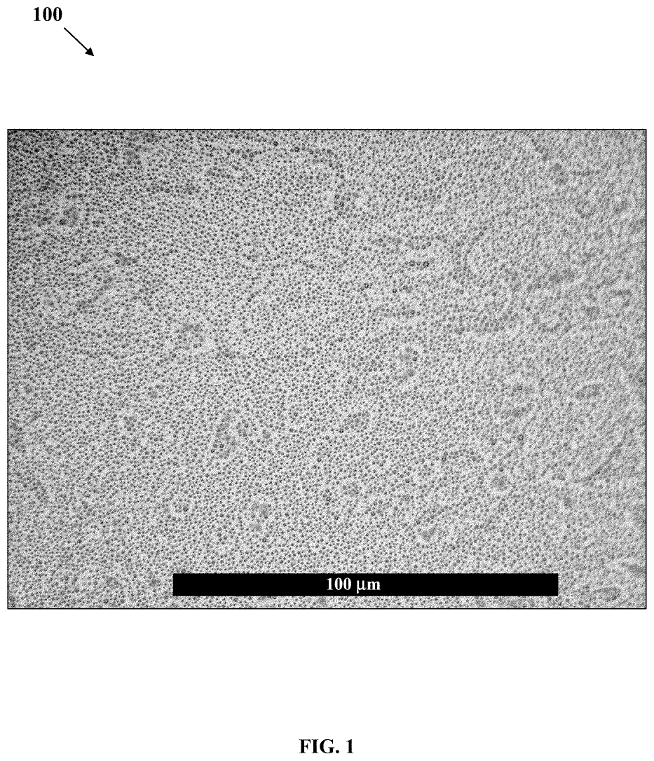

A biphasic coating with a 25 vol % continuous poly(ethylene glycol) (PEG) phase and a 75 vol % discrete structural poly(tetrahydrofuran) (pTHF) phase is formed as follows. The 25/75 PEG/pTHF is prepared by adding PEG 600 (5.00 g), pTHF 650 (13.10 g), dibutyltin dilaurate (0.058 g, about 2000 ppm), and 2-butanone (29.09 g) into a mixer cup followed by centrifugal mixing for one minute at 2000 revolutions per minute (RPM). Desmodur 3300 (10.99 g) is added and the solution is mixed for one minute at 2000 RPM. The resulting solution is sprayed onto aluminum with an LPH-80 Anest Iwata HVLP spray gun in 4 passes (30 seconds between passes). The film is allowed to cure overnight at room temperature (about 25° C.). The cured film is approximately 100 microns thick. The structure of the coating is shown in FIG. 1.

FIG. 1 is an optical microscope image of the biphasic polymer structure containing 25 vol % PEG and 75 vol % pTHF coating. The darker spots in FIG. 1 are the poly(tetrahydrofuran) discrete structural phase and the clear continuous phase is the poly(ethylene glycol) transport phase.

Incorporation of Chemical Active into Coating

Twelve samples (about 5.1 cm×5.1 cm) were cut out of each coating and submerged in solutions of 10 wt %, 1 wt %, 0.1 wt %, and 0 wt % of analyte (alkyldimethylbenzylammonium chloride) in deionized water and allowed to soak overnight. The solution of 0 wt % corresponds to pure deionized water. Films are removed from the solution, rinsed with deionized water, blotted dry on a Kimwipe®, and placed on a clean glass surface. Samples are measured after removal from the solution.

### Impedance Measurements to Determine Concentration of Chemical Active

The impedance of analyte-filled biphasic polymers is measured using electrochemical impedance spectroscopy (EIS). The calibration samples are disks (1.27 cm² area) cut from larger films (0.22 mm thickness). The samples are placed in a conductivity measurement fixture consisting of a large-diameter (1.91 cm) bottom stainless-steel electrode and limiting-area (1.11 cm diameter) top stainless-steel electrode pressed down onto the film with a spring. EIS spectra are measured using a Gamry 600+ potentiostat. Spectra are measured from 5,000,000 Hz to 0.1 Hz with a 10 mV amplitude sinusoidal waveform. Film resistance is determined from the spectra by fitting to an appropriate equivalent circuit model (Randles circuit).

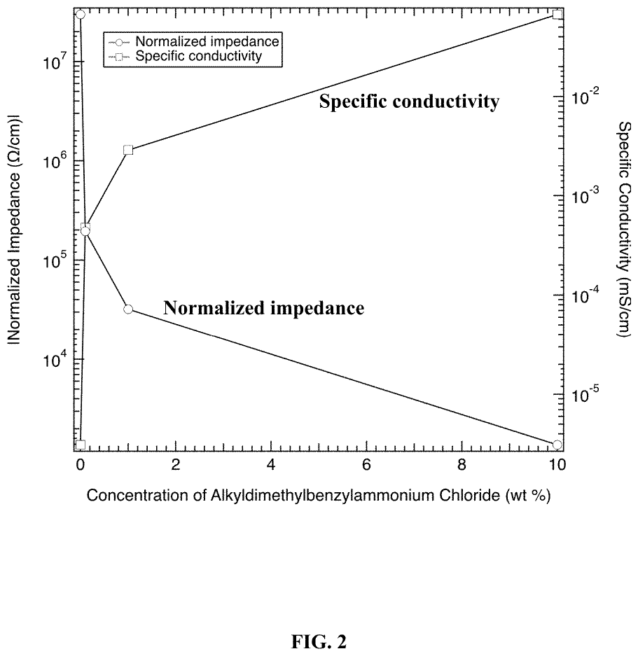

FIG. 2 is a plot of impedance, normalized to the film thickness, and specific conductivity measured as a function of concentration of chemical active (alkyldimethylbenzylammonium chloride). Note that specific conductivity is specific conductance. This data can be used to establish a look-up table, or fit to a curve to establish an equation, and used to determine an unknown concentration of chemical active from an impedance measurement.

In this detailed description, reference has been made to multiple embodiments and to the accompanying drawings in which are shown by way of illustration specific exemplary embodiments of the invention. These embodiments are described in sufficient detail to enable those skilled in the art to practice the invention, and it is to be understood that modifications to the various disclosed embodiments may be made by a skilled artisan.

Where methods and steps described above indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

All publications, patents, and patent applications cited in this specification are herein incorporated by reference in their entirety as if each publication, patent, or patent application were specifically and individually put forth herein.

The embodiments, variations, and figures described above should provide an indication of the utility and versatility of the present invention. Other embodiments that do not provide all of the features and advantages set forth herein may also be utilized, without departing from the spirit and scope of the present invention. Such modifications and variations are considered to be within the scope of the invention defined by the claims.

What is claimed is:

1. A system for sensing a chemical active in a coating, said system comprising:
a coating disposed on a substrate;
a chemical active contained within said coating, wherein said chemical active is mobile within said coating, and wherein said chemical active is ionically and/or electrically conductive;
a first electrode and a second electrode configured to measure AC impedance within said coating; and
an electrical meter configured in electrical communication with said first and second electrodes to read a signal corresponding to said AC impedance.

2. The system of claim 1, wherein said coating is a polymer.

3. The system of claim 2, wherein said polymer contains at least a first phase that is continuous and a second phase that is discrete or continuous.

4. The system of claim 3, wherein said first phase and said second phase are phase-separated on an average length scale of phase separation selected from about 10 nanometers to about 1 millimeter.

5. The system of claim 1, wherein said chemical active is present in said coating in a concentration from about 0.001 wt % to about 25 wt %.

6. The system of claim 1, wherein said chemical active is characterized by a diffusivity from about 10⁻¹⁸ m²/s to about 10⁻⁹ m²/s measured at 25° C.

7. The system of claim 1, wherein said chemical active is a liquid or is dissolved in a solvent.

8. The system of claim 1, wherein said chemical active is selected from the group consisting of a salt, an acid, a base, an oxidizer, an ionizable compound, an ionic liquid, and combinations thereof.

9. The system of claim 8, wherein said chemical active is a quaternary ammonium salt.

10. The system of claim 1, wherein said first electrode and said second electrode are each pressed against an external surface of said coating, and wherein there is a region of said coating interposed between said first electrode and said second electrode

11. The system of claim 1, wherein one of said first electrode and said second electrode is disposed at or near an external surface of said coating, and wherein the other of said first electrode and said second electrode is distally disposed at the opposite side of said coating.

12. The system of claim 1, wherein one of said first electrode and said second electrode is pressed against an external surface of said coating, and wherein the other of said first electrode and said second electrode is at least partially embedded within said coating.

13. The system of claim 1, wherein said first electrode and said second electrode are each at least partially embedded within said coating.

14. The system of claim 1, wherein said first electrode and said second electrode are each in the form of concentric circles.

15. The system of claim 1, wherein said first electrode and said second electrode are each in the form of parallel lines or parallel shapes.

16. The system of claim 1, wherein said electrical meter is configured to apply an AC waveform to said first electrode and said second electrode.

17. The system of claim 1, wherein said electrical meter is configured to sense at a single frequency.

18. The system of claim 1, wherein said electrical meter is configured to sense at multiple frequencies.

19. A method of measuring the concentration of a chemical active in a coating, said method comprising:
(a) providing a system comprising: a coating disposed on a substrate; a chemical active contained within said coating, wherein said chemical active is mobile within said coating, and wherein said chemical active is ionically and/or electrically conductive; a first electrode and a second electrode configured to measure AC impedance within said coating; and an electrical meter configured in electrical communication with said first and second electrodes to read a signal corresponding to said AC impedance;
(b) optionally, calibrating said system with known conductivity samples;
(c) optionally, wetting a surface of said coating;
(d) pressing said first electrode and said second electrode against said coating;
(e) reading out an impedance value; and
(f) converting said impedance value to a concentration of said chemical active.

20. The method of claim 19, wherein said chemical active is a liquid or is dissolved in a solvent.

21. The method of claim 19, wherein said chemical active is selected from the group consisting of a salt, an acid, a base, an oxidizer, an ionizable compound, an ionic liquid, and combinations thereof.

22. The method of claim 19, wherein step (c) is performed and utilizes an aqueous solvent to wet said surface of said coating.

23. A method of measuring the concentration of a chemical active in a coating, said method comprising:
(a) providing a system comprising: a coating disposed on a substrate; a chemical active contained within said coating, wherein said chemical active is mobile within said coating, and wherein said chemical active is ionically and/or electrically conductive; a first electrode and a second electrode configured to measure AC impedance within said coating; and an electrical meter configured in electrical communication with said first and second electrodes to read a signal corresponding to said AC impedance;
(b) optionally, calibrating said system with known conductivity samples;
(c) adding one or more droplets of a solvent to a surface of said coating;
(d) waiting a selected amount of time;
(e) pressing said first electrode and said second electrode against a surface region comprising said solvent and said coating;
(f) reading out an impedance value; and
(g) converting said impedance value to a concentration of said chemical active.

24. The method of claim 23, wherein said chemical active is selected from the group consisting of a salt, an acid, a base, an oxidizer, an ionizable compound, an ionic liquid, and combinations thereof.

25. The method of claim 23, wherein said solvent in step (c) is an aqueous solvent.
